(19) United States
(12) Patent Application Publication
(10) Pub. No.: US 2026/0058006 A1
(43) Pub. Date: Feb. 26, 2026

(54) SYSTEMS, METHODS, AND DEVICES FOR DENTAL IMAGING DEVICE HEALTH MONITORING

(71) Applicant: SOTA CLOUD CORPORATION, Orange, CA (US)

(72) Inventor: Dustin JOHNSON, Fullerton, CA (US)

(21) Appl. No.: 19/305,166

(22) Filed: Aug. 20, 2025

Related U.S. Application Data
(60) Provisional application No. 63/686,622, filed on Aug. 23, 2024.

(51) Int. Cl. G16H 40/40; G06F 11/07

(52) U.S. Cl. CPC G16H 40/40; G06F 11/0733; G06F 11/079; G06F 11/0793

(57) ABSTRACT

Monitoring and reporting the health of dental imaging devices, includes an interface configured to integrate with dental imaging devices using one or more application programming interfaces (APIs), a monitoring module configured to capture and log device status codes, error codes, device location, and operational data during use of the dental imaging devices, a database configured to store the captured and logged data, a backend server configured to process status data received from the monitoring module and generate processed data for storage in the database, and a reporting module configured to provide stakeholders with insights into operational health of the devices based on the processed data stored in the database.

### CROSS-REFERENCE TO RELATED APPLICATIONS

This application claims priority to U.S. Application No. 63/686,622 filed August 23, 2024, which is hereby incorporated by reference in its entirety.

### FIELD OF INVENTION

The present disclosure relates to dental imaging systems, and more particularly to systems and methods for monitoring the health and operational status of dental imaging devices.

### BACKGROUND

Dental imaging devices have become fundamental tools in modern dental practices, enabling practitioners to diagnose and treat various dental conditions through advanced imaging technologies. These devices encompass a wide range of equipment including intraoral X-ray sensors, intraoral cameras, panoramic X-ray machines, cephalometric X-ray machines, Cone Beam Computed Tomography (CBCT) machines, and intraoral scanners. Each type of device serves specific diagnostic purposes and integrates with dental practice management software through various interfaces such as software development kits (SDKs), TWAIN protocols, DirectShow, Universal Video Class (UVC), and other application programming interfaces (APIs).

The expansion of Dental Support Organizations (DSOs) has led to the management of dental imaging equipment across multiple locations and on a larger scale than traditional single-practice operations. DSOs may oversee thousands of devices distributed across numerous dental offices, creating complex operational environments where equipment monitoring and maintenance become increasingly challenging. The diversity of device brands, models, and ages within these networks adds layers of complexity to equipment management.

Traditional approaches to monitoring dental imaging device performance typically rely on localized logging systems that store operational data on individual computers connected to each device. This decentralized approach can create information silos where device status, error logs, and performance data remain isolated at individual workstations. Such fragmentation can hinder comprehensive oversight of equipment health across multiple locations and may delay the identification of emerging issues or patterns that could indicate broader systemic problems.

IT teams responsible for maintaining dental imaging equipment often face challenges related to the specialized nature of these devices. The technical knowledge required to troubleshoot imaging equipment may differ from general IT support expertise, potentially leading to longer resolution times when issues arise. Additionally, the variety of device manufacturers and models within a single organization can require familiarity with multiple different interfaces, error codes, and troubleshooting procedures.

The operational efficiency of dental practices depends heavily on the reliable performance of imaging equipment. Device downtime can disrupt patient care workflows, delay diagnoses, and impact practice productivity. Proactive monitoring and maintenance approaches that can identify potential issues before they result in equipment failure would provide value in maintaining continuous operations and minimizing disruptions to patient care.

### SUMMARY

This summary is provided to introduce a selection of concepts in a simplified form that are further described below in the detailed description. This summary is not intended to identify key features or essential features of the claimed subject matter, nor is it intended to be used as an aid in determining the scope of the claimed subject matter.

According to an aspect of the present disclosure, a system for monitoring and reporting the health of dental imaging devices is provided. The system includes an interface configured to integrate with one or more application programming interfaces (APIs). A monitoring module is configured to capture and log device status codes, error codes, device location, and operational data during use of the dental imaging devices. A database is configured to store the captured and logged data. A backend server is configured to process status data received from the monitoring module and generate processed data for storage in the database. A reporting module is configured to provide stakeholders with insights into operational health of the dental imaging devices based on the processed data stored in the database and display the insights on a user interface of an electronic display.

According to another aspect of the present disclosure, a method of monitoring and reporting the health of dental imaging devices is provided. The method includes integrating a monitoring system with dental imaging devices using one or more application programming interfaces (APIs). Operational data including status codes, error codes, user interactions, device location, and device metadata during operation of the dental imaging devices is captured and logged. The captured operational data is transmitted to a backend server. The operational data is processed at the backend server to generate standardized data. The standardized data is stored in a database. The stored data is analyzed. Insights into device health and troubleshooting information are provided on a user interface.

According to another aspect of the present disclosure, a non-transitory computer-readable medium storing instructions is provided that, when executed by a processor, cause the processor to perform operations including integrating a monitoring system with dental imaging devices using one or more application programming interfaces (APIs). Operational data including status codes, error codes, user interactions, device location, and device metadata during operation of the dental imaging devices is captured and logged. The captured operational data is transmitted to a backend server. The operational data is processed at the backend server to generate standardized data. The standardized data is stored in a database. The stored data is analyzed. Insights into device health and troubleshooting information are provided on a user interface.

The foregoing general description of the illustrative embodiments and the following detailed description thereof are merely exemplary aspects of the teachings of this disclosure and are not restrictive.

### BRIEF DESCRIPTION OF FIGURES

Non-limiting and non-exhaustive examples are described with reference to the following figures.

FIG. 1 is a block diagram of a system and the flow for monitoring the health of a dental imaging device, according to an embodiment.

FIG. 2 is a block diagram of a system and the flow for monitoring the health of a dental imaging device, according to another embodiment that includes a plugin module to monitor device status.

FIG. 3 is a flow chart of a process for monitoring the health of a dental imaging device, according to an embodiment.

FIG. 4 is a block diagram of a notification system, consistent with embodiments.

### DETAILED DESCRIPTION

The following description sets forth exemplary aspects of the present disclosure. It should be recognized, however, that such description is not intended as a limitation on the scope of the present disclosure. Rather, the description also encompasses combinations and modifications to those exemplary aspects described herein.

A detailed description of systems, devices, and methods consistent with embodiments of the present disclosure is provided below. While several embodiments are described, it should be understood that disclosure is not limited to any one embodiment, but instead encompasses numerous alternatives, modifications, and equivalents. In addition, while numerous specific details are set forth in the following description in order to provide a thorough understanding of the embodiments disclosed herein, some embodiments can be practiced without some or all of these details. Moreover, for the purpose of clarity, certain technical material that is known in the related art has not been described in detail in order to avoid unnecessarily obscuring the disclosure.

The dental imaging device health monitoring system of the subject technology provides comprehensive oversight of dental imaging equipment across various practice environments. Current approaches to device health monitoring in dental practices may lack the comprehensive data aggregation and analysis capabilities that could provide insights into usage patterns, error trends, and predictive maintenance opportunities. The integration of advanced analytics and machine learning techniques into device monitoring systems represents an area where technological advancement could enhance equipment management practices.

In some cases, the system may monitor multiple types of dental imaging devices simultaneously, including intraoral X-ray sensors, intraoral cameras, panoramic X-ray machines, cephalometric X-ray machines, cone beam computed tomography machines, and intraoral scanners. The system may integrate with existing dental practice workflows while providing enhanced visibility into device performance and operational status. In some cases, the system may operate across multiple locations within large dental support organizations, enabling centralized monitoring of thousands of devices distributed across numerous practice sites.

The system may capture and analyze device status information through various integration methods, including native software development kits, Technology Without an Interesting Name (TWAIN) interfaces, DirectShow protocols, Universal Video Class interfaces, and other application programming interfaces. In some cases, the system may collect contextual information surrounding device operations, such as user interactions, error conditions, performance metrics, and environmental factors that may influence device functionality. The collected data may be processed and standardized to enable consistent analysis across different device brands, models, and operational environments. The system may provide stakeholders with actionable insights regarding device health, usage patterns, and potential maintenance requirements.

Data storage and processing capabilities may accommodate both on-premise and cloud-hosted deployment scenarios, allowing organizations to select implementation approaches that align with their infrastructure preferences and security requirements. In some cases, the system may aggregate device information from multiple sources into centralized databases that support comprehensive analysis and reporting functions. The system may enable real-time monitoring of device status while maintaining historical records for trend analysis and predictive maintenance planning. Advanced analytics capabilities may include machine learning components that analyze historical performance data to identify patterns and predict potential device failures before they occur.

The system may provide automated troubleshooting assistance by correlating observed error conditions with known resolution procedures and maintenance recommendations. In some cases, stakeholders may receive detailed error context and guidance for addressing device issues, reducing the technical expertise required for effective troubleshooting. The system may support customizable notification mechanisms that alert appropriate personnel when device issues are detected, enabling proactive response to potential problems. Modular architecture design may allow for the addition of new device types and enhanced functionality as dental imaging technology continues to evolve.

### Example Architecture

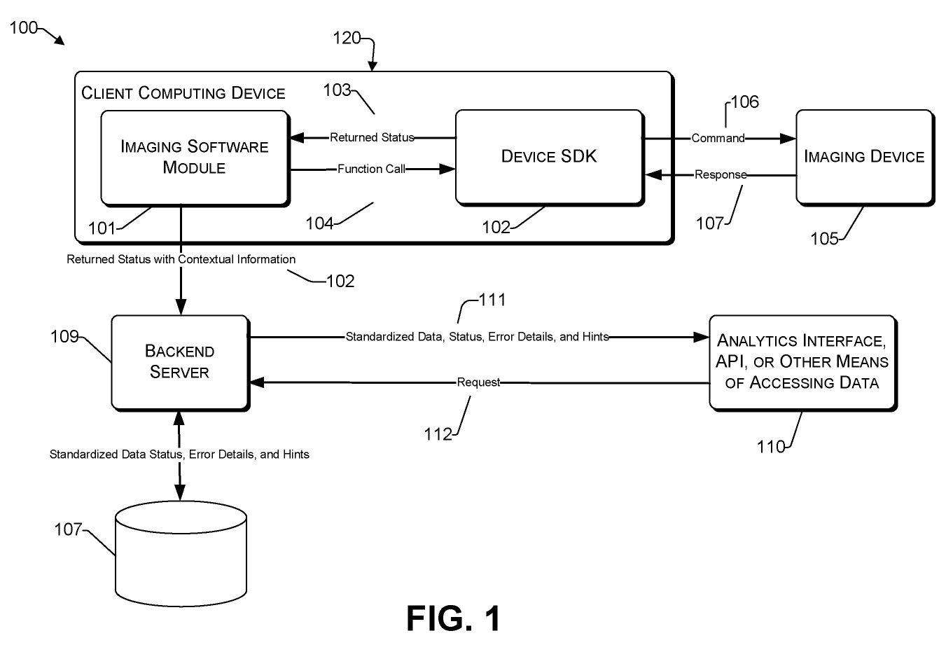

Referring to FIG. 1, a system 100 for monitoring the health of a dental imaging device is shown according to an embodiment. The system 100 includes an architecture comprises multiple interconnected components that work together to monitor and report the health of dental imaging devices. A client computer 120 serves as the primary interface point where dental imaging operations occur and houses the imaging software 101 along with a device interface 102. The client computer 120 may be a standard personal computer, workstation, or specialized dental computing device that provides the processing power and connectivity for device operations. An imaging device 105 connects to the client computer 120 through wired or wireless communication channels, enabling the capture and processing of dental images. The system architecture may support modular expansion to accommodate new device types and enhanced functionality as dental imaging technology continues to advance.

The imaging software 101 operates on the client computer 120 and provides the primary user interface for dental imaging operations. In some cases, the imaging software 101 may be a comprehensive dental practice management system or a specialized imaging application designed for specific device types. The imaging software 101 communicates with the device interface 102 through function calls 104 that initiate various device operations and retrieve status information. The device interface 102 may be one or more of software development kits, TWAIN interfaces, DirectShow protocols, Universal Video Class interfaces, or other application programming interfaces (APIs) that enable communication with the imaging device 105. The device interface 102 serves as a monitoring module that captures and logs device status codes, error codes, device location, and other relevant operational data during the use of the dental imaging devices.

The device interface 102 generates device commands 106 that are transmitted to the imaging device 105 to control various operational functions such as image acquisition, calibration, and status reporting. The imaging device 105 responds with device responses 107 that contain operational data, image information, and status indicators. In some cases, the imaging device 105 may be a two dimensional image generating device such as an intraoral X-ray sensor, intraoral camera, panoramic X-ray machine, or a cephalometric X-ray machine. In some embodiments, the imaging device 105 may be a three-dimensional image generating device such as a cone beam computed tomography (CBCT) machine, or intraoral scanner. The device interface 102 processes the device responses 107 and generates status codes 103 that indicate the operational state of the imaging device 105, including normal operation indicators and error conditions.

The system architecture includes a backend server 109 that receives status data 108 from the client computer 120 through network communication channels. The status data 108 comprises both the status codes 103 returned by the device interface 102 and contextual information collected from the imaging device 105, client computer 120, and operational environment. The backend server 109 may be located on the same premises as the client computer 120 for local deployment scenarios or may be hosted remotely in cloud-based data centers for distributed implementations. In some cases, the backend server 109 may support scalable architectures capable of monitoring thousands of devices across multiple locations with data aggregated into a central processing system. The backend server 109 processes the status data 108 and generates processed data 114 that is formatted and standardized for storage and analysis purposes.

A database system 113 stores the processed data 114 received from the backend server 109, providing persistent storage for captured and logged data. The database system 113 may be hosted locally on the same premises as the client computer 120 or may be deployed in cloud-based environments depending on organizational preferences and infrastructure requirements. In some cases, the database system 113 may comprise relational database management systems, non-relational database systems, or hybrid storage architectures that accommodate different data types and access patterns. The database system 113 may store historical data for long-term analysis of device health and performance trends, enabling comprehensive tracking of device operations over extended time periods.

A user interface 110 provides stakeholders with access to the stored device information through various presentation formats. The user interface 110 may comprise analytics dashboards, web-based interfaces, mobile applications, or application programming interfaces that enable different types of access to the stored data. The user interface 110 generates interface requests 112 that are transmitted to the backend server 109 to retrieve specific information from the database system 113. The backend server 109 processes the interface requests 112 and returns standardized data 111 that is formatted according to the requirements of the requesting interface type. In some cases, the user interface 110 may serve as a reporting module that provides stakeholders with insights into the operational health of the devices, including device status information, error history, and user interaction patterns. The user interface 110 may also function as a troubleshooting module that utilizes the logged data to assist stakeholders in diagnosing and resolving device issues, including providing detailed error context and guidance for resolving known errors.

The device interface 102 may accommodate multiple integration methods to support comprehensive compatibility with various dental imaging devices and communication protocols. In some cases, the device interface 102 may utilize native software development kits provided by device manufacturers to establish direct communication channels with the imaging device 105. Native software development kit integration may provide access to device-specific functions, advanced operational parameters, and proprietary features that enable enhanced monitoring capabilities. The device interface 102 may also support TWAIN protocol integration, which provides standardized communication methods for image acquisition devices across different manufacturers and device types. TWAIN integration may enable the device interface 102 to communicate with legacy devices and systems that utilize established imaging standards, ensuring backward compatibility with existing dental practice infrastructure.

DirectShow protocol support within the device interface 102 may facilitate integration with video-based dental imaging devices such as intraoral cameras and real-time imaging systems. In some cases, DirectShow integration may enable the device interface 102 to capture streaming video data, monitor device performance during continuous operation, and log operational status information for video-based imaging procedures. Universal Video Class integration may provide additional compatibility with standardized video devices that conform to USB video specifications. The device interface 102 may support other application programming interfaces and communication protocols to accommodate emerging technologies and specialized device implementations that may not conform to standard integration methods.

The device interface 102 may generate a function call 104 to initiate communication with the imaging device 105 through the selected integration method. The function call 104 may contain operational parameters, device commands, and configuration information that specify the desired device operation or status inquiry. In some cases, the function call 104 may request device identification information, operational status data, error condition reports, or image acquisition parameters depending on the monitoring requirements and device capabilities. The device interface 102 may process the function call 104 according to the communication protocol specifications and device interface requirements to ensure proper formatting and transmission to the imaging device 105.

The imaging device 105 may support various dental imaging modalities including intraoral X-ray sensors that capture digital radiographic images of individual teeth and surrounding structures. Intraoral camera devices may provide real-time visual imaging capabilities for diagnostic and patient education purposes, enabling direct observation of oral conditions and treatment areas. Panoramic X-ray machines may generate comprehensive images of the entire dental arch and surrounding anatomical structures through rotational imaging techniques. Cephalometric X-ray machines may produce lateral and frontal skull images for orthodontic analysis and treatment planning purposes. Cone beam computed tomography machines may generate three-dimensional volumetric images of dental and maxillofacial structures with enhanced spatial resolution and diagnostic detail. Intraoral scanners may capture three-dimensional surface geometry of dental structures for digital impression applications and computer-aided design workflows.

The device interface 102 may transmit a device command 106 to the imaging device 105 based on the parameters specified in the function call 104. The device command 106 may contain low-level control instructions, operational parameters, and configuration settings that direct the imaging device 105 to perform specific functions or report status information. In some cases, the device command 106 may initiate image acquisition sequences, calibration procedures, diagnostic tests, or status reporting functions depending on the monitoring requirements and device capabilities. The imaging device 105 may process the device command 106 according to the device-specific operational protocols and generate a device response 107 that contains the requested information or operational results. The device response 107 may include operational status indicators, error condition reports, device identification data, performance metrics, and image data depending on the nature of the device command 106 and the capabilities of the imaging device 105.

The device interface 102 generates a status code 103 that represents the operational state and performance characteristics of the imaging device 105 following the execution of the function call 104. The status code 103 may contain numerical indicators, text-based messages, or structured data elements that convey information about device functionality, error conditions, or operational parameters. In some cases, the status code 103 may include device-specific error codes that correspond to manufacturer-defined diagnostic categories, enabling precise identification of operational issues or performance anomalies. The status code 103 may also contain success indicators that confirm normal device operation and validate the completion of requested imaging procedures or diagnostic functions.

The device interface 102 processes the status code 103 and combines this information with additional contextual data to generate the status data 108 that provides comprehensive operational documentation. The status data 108 may include specific contextual information such as device brand identification, model designation, and serial number details that uniquely identify the imaging device 105 within the monitoring system. In some cases, the status data 108 may incorporate PC name information that identifies the client computer 120 hosting the device interface 102, along with practice ID and organization ID parameters that establish the geographic and administrative context for the device operation. The status data 108 may contain user ID information that identifies the operator who initiated the imaging procedure, along with status type classifications that categorize the nature of the operational event.

The status data 108 may include error type indicators that classify detected issues according to predefined categories such as communication failures, hardware malfunctions, or software compatibility problems. The status data 108 may contain error text descriptions that provide human-readable explanations of detected conditions, along with exception details that capture technical diagnostic information for troubleshooting purposes. In some cases, the status data 108 may include error hints that offer specific guidance for resolving identified issues, along with software version information that documents the operational environment and compatibility parameters. The device interface 102 may format the status data 108 according to standardized protocols that enable consistent processing across different device types and communication interfaces.

The status data 108 may be transmitted from the client computer 120 to the backend server 109 through network communication channels that support reliable data transfer and error handling capabilities. The backend server 109 receives the status data 108 and performs validation procedures to ensure data integrity and completeness before initiating processing operations. In some cases, the backend server 109 may authenticate the source of the status data 108 and verify that the transmitting client computer 120 has appropriate authorization to submit device monitoring information. The backend server 109 may apply data transformation algorithms that standardize the format and structure of the received status data 108 to enable consistent storage and analysis procedures across different device types and operational environments.

The backend server 109 processes the status data 108 through standardization procedures that convert device-specific information into uniform data formats suitable for database storage and analytical processing. The backend server 109 may parse the contextual information contained within the status data 108 and map device brand, model, and serial number details to standardized equipment databases that maintain comprehensive device specifications and operational parameters. In some cases, the backend server 109 may correlate PC name, practice ID, and organization ID information with geographic databases that provide location context and administrative hierarchy details for reporting and analysis purposes. The backend server 109 may process user ID information to establish operator accountability and usage pattern tracking capabilities that support performance monitoring and training requirements.

The backend server 109 generates the processed data 114 through transformation algorithms that structure the standardized information for optimal storage and retrieval performance within the database system 113. The processed data 114 may include normalized data elements that eliminate redundancy and ensure referential integrity across related database tables and data structures. In some cases, the processed data 114 may incorporate calculated fields that derive performance metrics, trend indicators, or statistical summaries from the raw operational data contained within the status data 108. The processed data 114 may include timestamp information that establishes precise chronological ordering of operational events, along with data quality indicators that assess the completeness and reliability of the captured information.

The user interface 110 may generate an interface request 112 that specifies the particular data elements, filtering criteria, and presentation formats required for stakeholder access to the stored device information. The interface request 112 may contain query parameters that define the scope of data retrieval operations, including date ranges, device types, location filters, or error condition categories that focus the analysis on specific operational aspects. In some cases, the interface request 112 may specify aggregation functions that calculate summary statistics, trend analyses, or comparative metrics across multiple devices or time periods. The backend server 109 processes the interface request 112 by executing database queries against the stored processed data 114 and applying the specified filtering and formatting operations to generate the standardized data 111 that meets the requirements of the requesting user interface 110.

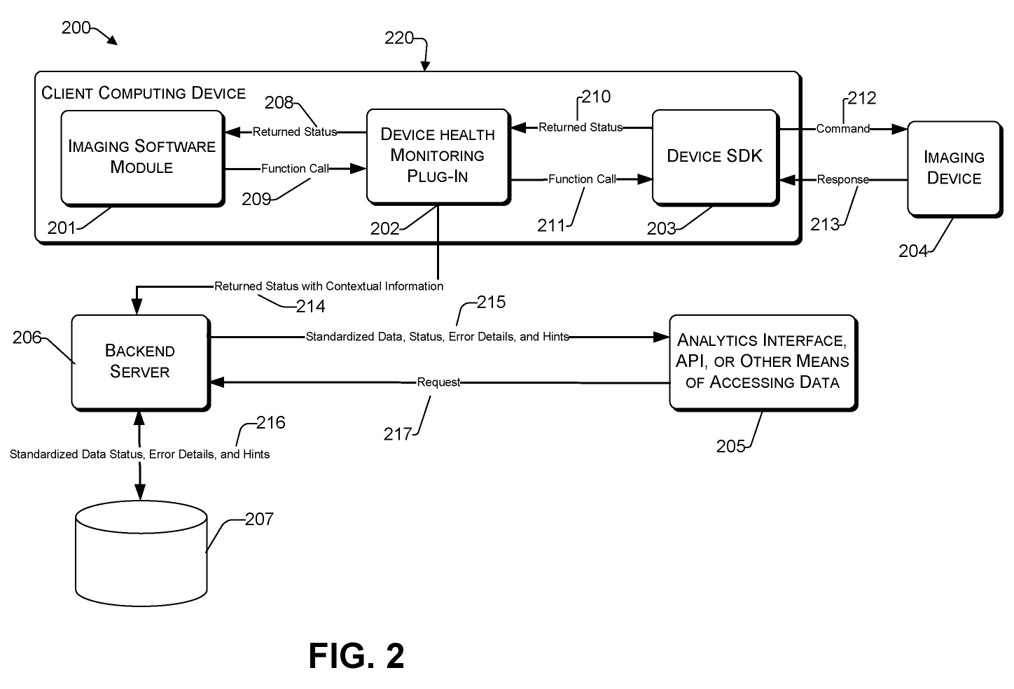

Referring to FIG. 2, a system 200 for monitoring dental imaging device health is shown according to another embodiment. The system 200 is similar to the system 100 except that the system 200 includes a device health monitoring plugin module 202 that provides monitoring capabilities for existing dental imaging systems without requiring modifications to established software environments. The device health monitoring plugin 202 may operate as an intermediary component that intercepts communication between a dental imaging software 201 and a device SDK 203, enabling comprehensive device monitoring while maintaining compatibility with existing operational workflows. In some cases, the device health monitoring plugin 202 may be implemented as a bolt-on solution that integrates with established dental practice systems through standardized software interfaces and communication protocols. The device health monitoring plugin 202 may support various integration methods including stub libraries, dynamic link library interception, or application programming interface wrapping techniques that enable transparent monitoring without disrupting normal device operations.

The dental imaging software 201 operates as the primary user interface and control system for dental imaging operations, providing practitioners with familiar operational procedures and established workflows. The dental imaging software 201 may comprise existing practice management systems, specialized imaging applications, or integrated dental software platforms that have been deployed within dental practice environments. In some cases, the dental imaging software 201 may generate function calls intended for direct communication with the device SDK 203, but these communications may be intercepted by the device health monitoring plugin 202 through various interception mechanisms. The dental imaging software 201 may continue to operate according to established procedures while the device health monitoring plugin 202 captures operational data and device status information transparently.

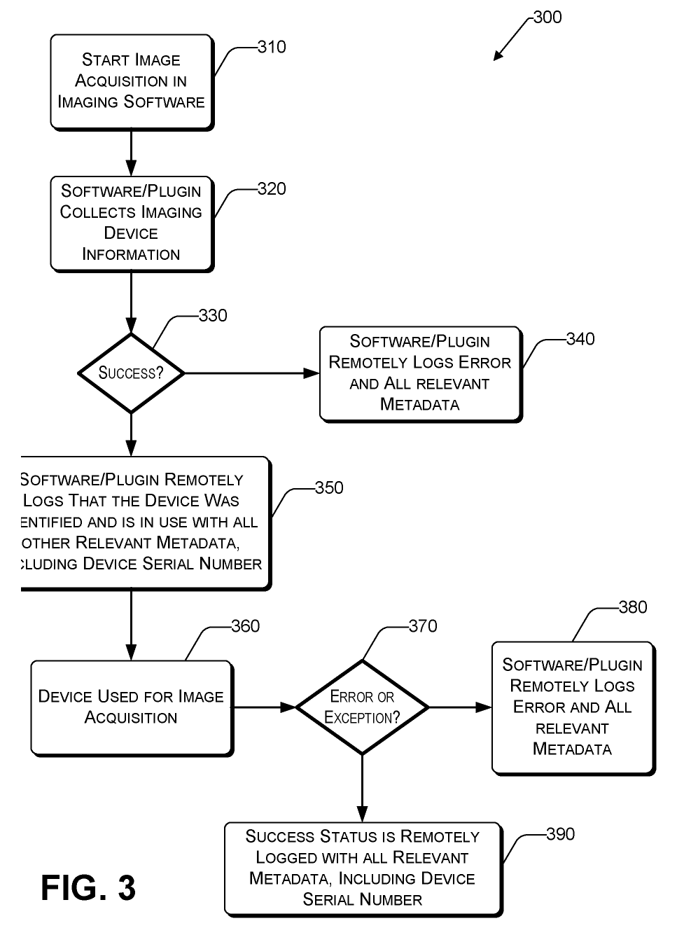

With continued reference to FIG. 3, the device SDK 203 provides the underlying communication interface between software systems and a dental imaging device 204, enabling control functions, status reporting, and data transfer operations. The device SDK 203 may comprise manufacturer-provided software libraries, device drivers, or application programming interfaces that enable direct hardware communication and device control capabilities. In some cases, the device SDK 203 may support various communication protocols including native manufacturer interfaces, TWAIN implementations, DirectShow protocols, or Universal Video Class specifications depending on the device type and manufacturer requirements. The device SDK 203 may process device function calls 211 received from the device health monitoring plugin 202 and generate appropriate device commands 212 that control the operational functions of the dental imaging device 204.

The dental imaging device 204 may represent various types of dental imaging equipment including intraoral X-ray sensors, intraoral cameras, panoramic X-ray machines, cephalometric X-ray machines, cone beam computed tomography machines, or intraoral scanners that require software control and monitoring capabilities. The dental imaging device 204 may respond to device commands 212 received from the device SDK 203 by executing the requested operations and generating device responses 213 that contain operational results, status information, or error conditions. In some cases, the dental imaging device 204 may provide real-time status updates, diagnostic information, or performance metrics through the device responses 213 that enable comprehensive monitoring of device health and operational characteristics. The dental imaging device 204 may support various communication interfaces including wired connections, wireless protocols, or network-based communication methods that enable reliable data transfer and control operations.

As further shown in FIG. 3, the device health monitoring plugin 202 may intercept function calls generated by the dental imaging software 201 through an intercepted function call 209 mechanism that captures the intended communication before transmission to the device SDK 203. The intercepted function call 209 may contain operational parameters, device commands, or configuration information that the dental imaging software 201 intended to transmit directly to the device SDK 203. In some cases, the device health monitoring plugin 202 may analyze the intercepted function call 209 to extract contextual information about the requested operation, user interactions, or device configuration parameters that support comprehensive monitoring capabilities. The device health monitoring plugin 202 may then forward the intercepted function call 209 to the device SDK 203 as a device function call 211 that maintains the original operational intent while enabling monitoring data collection.

The device health monitoring plugin 202 may implement interception mechanisms through stub libraries that replace or wrap the original device SDK 203 interfaces with monitoring-enabled equivalents that capture operational data while maintaining functional compatibility. Stub library implementations may provide identical function signatures and return values as the original device SDK 203 interfaces, ensuring that the dental imaging software 201 continues to operate without modification or compatibility issues. In some cases, the device health monitoring plugin 202 may utilize dynamic link library interception techniques that redirect function calls at runtime, enabling monitoring capabilities without requiring changes to the dental imaging software 201 or device SDK 203 components. The device health monitoring plugin 202 may also implement application programming interface wrapping methods that encapsulate the original device SDK 203 functions within monitoring-enabled wrapper functions that collect operational data before and after device operations.

With continued reference to FIG. 3, the device SDK 203 processes the device function call 211 received from the device health monitoring plugin 202 and generates status codes 210 that indicate the operational results, error conditions, or device status information resulting from the requested operation. The status codes 210 may contain device-specific error indicators, success confirmations, or operational parameters that provide detailed information about the device operation results. In some cases, the status codes 210 may include timing information, performance metrics, or diagnostic data that enable comprehensive assessment of device health and operational characteristics. The device health monitoring plugin 202 may capture the status codes 210 returned by the device SDK 203 and process this information to generate both monitoring data and response information for the dental imaging software 201.

The device health monitoring plugin 202 may generate a status return signal 208 that provides the dental imaging software 201 with the operational results and status information that would normally be received directly from the device SDK 203. The status return signal 208 may maintain identical format and content characteristics as the original device SDK 203 responses, ensuring that the dental imaging software 201 receives expected information without detecting the presence of the monitoring plugin. In some cases, the status return signal 208 may include additional error handling or status enhancement features that improve the reliability or functionality of the device communication while maintaining compatibility with existing software expectations. The device health monitoring plugin 202 may simultaneously utilize the captured status information to generate status data 214 that contains comprehensive monitoring information for transmission to a backend server 206.

As further shown in FIG. 3, the device health monitoring plugin 202 may compile the status data 214 by combining the status codes 210 received from the device SDK 203 with contextual information collected from the operational environment, user interactions, and device characteristics. The status data 214 may include device brand identification, model specifications, serial number details, PC name information, practice ID parameters, organization ID data, user ID information, status type classifications, error type indicators, error text descriptions, exception details, error hints, and software version information that provide comprehensive operational documentation. In some cases, the status data 214 may incorporate timing information, performance metrics, or usage pattern data that enable advanced analytics and predictive maintenance capabilities. The device health monitoring plugin 202 may format the status data 214 according to standardized protocols that enable consistent processing and analysis across different device types and operational environments.

The database system 113 provides comprehensive data storage capabilities that accommodate diverse deployment scenarios and organizational infrastructure requirements. The database system 113 may support both on-premise implementations where data storage occurs within the dental practice's local computing environment and cloud-hosted configurations where data resides in remote data centers managed by cloud service providers. In some cases, on-premise database deployments may utilize local server hardware, network-attached storage systems, or dedicated database appliances that provide direct organizational control over data security, access policies, and backup procedures. Cloud-hosted database implementations may leverage distributed storage architectures, managed database services, or hybrid cloud configurations that provide scalability, redundancy, and geographic distribution capabilities while reducing local infrastructure management requirements.

### Example Methodology

Referring to FIG. 3, a method 300 for monitoring dental imaging devices is shown according to an embodiment. The method 300 illustrates the comprehensive process for capturing and logging device operational data throughout the image acquisition workflow. The method 300 provides a systematic approach for monitoring dental imaging device performance and collecting contextual information that supports troubleshooting and maintenance activities. In some cases, the method 300 may be implemented within the imaging software 101 or through specialized monitoring components that integrate with existing dental practice workflows. The method 300 may capture detailed operational data from multiple device types while maintaining compatibility with various integration protocols and communication interfaces.

The method 300 begins with a step 310 where a user initiates the image acquisition process within the dental imaging software environment. During the step 310, the user may select specific imaging parameters, configure device settings, or initiate diagnostic procedures depending on the clinical requirements and device capabilities. In some cases, the step 310 may involve user authentication procedures, patient identification processes, or treatment planning activities that establish the operational context for subsequent device monitoring activities. The step 310 may capture user identification information, including the signed-in user operating the device, which becomes part of the contextual data collected during the monitoring process. The method 300 may record the identifier of the PC to which the device is connected, establishing the hardware environment context for the imaging operation.

At step 320, the imaging software 101 or monitoring plugin 202 attempts to collect basic device information through the device interface 102. The step 320 may involve generating the function call 104 to retrieve device-specific parameters such as device brand, model designation, and the serial number of the device and any related interface devices. In some cases, the step 320 may collect environmental contextual information including PC specifications, software version details, practice identification data, and organizational information that provides comprehensive operational context. The step 320 may utilize native software development kits, TWAIN interfaces, DirectShow protocols, Universal Video Class interfaces, or other relevant application programming interfaces to establish communication with the imaging device 105. The step 320 may capture the location of the device within the practice environment, enabling geographic tracking and maintenance scheduling capabilities.

At step 330 that evaluates whether the device information collection process completed successfully during the step 320. The step 330 may analyze the device responses 107 received from the imaging device 105 to determine if communication was established and basic device parameters were retrieved without errors. In some cases, the step 330 may evaluate communication timeouts, protocol compatibility issues, or device availability conditions that could prevent successful information collection. The step 330 may assess the completeness and validity of the collected device information to ensure that subsequent monitoring activities can proceed with adequate operational context. The step 330 may determine the type and criticality of any errors encountered during the device information collection process.

When the step 330 determines that device information collection was unsuccessful, the method 300 proceeds to a step 340 where error information is logged to the backend server 109. The step 340 may capture the error or status code retrieved from the device's SDK 230 or API, along with comprehensive contextual information that supports troubleshooting activities. In some cases, the step 340 may record a full stack trace of any errors encountered during the device communication process, providing detailed diagnostic information for technical support personnel. The step 340 may generate a detailed hint or instruction for resolving errors based on the specific error conditions and device characteristics. The step 340 may compile the status data 108 that includes device brand information, model specifications, practice ID, organization ID, user ID, status type indicators, error type classifications, error text descriptions, exception details, and software version information.

When the step 330 determines that device information collection was successful, the method 300 proceeds to a step 350 where successful device identification is logged to the backend server 109. The step 350 may record that the imaging device 105 was identified successfully and is available for imaging operations, along with comprehensive contextual information that documents the operational environment. In some cases, the step 350 may capture device serial numbers, PC name information, user identification data, and location details that establish the operational context for subsequent imaging activities. The step 350 may generate the status data 108 that includes device brand, model, serial number, PC name, practice ID, organization ID, user ID, and software version information to provide complete operational documentation. The step 350 may record status type indicators that classify the operational state and error type classifications that indicate normal operation conditions.

At step 360, the imaging device 105 is used for image acquisition operations through the device interface 102. The step 360 may involve transmitting the device commands 106 to control imaging parameters, initiate capture sequences, or configure device settings according to the clinical requirements. In some cases, the step 360 may execute multiple device operations including calibration procedures, exposure settings, image processing functions, or data transfer activities depending on the imaging modality and device capabilities. The step 360 may monitor device performance during active imaging operations, capturing operational metrics and performance indicators that support device health assessment. The step 360 may continue to collect contextual information including user interactions, device location data, and operational parameters throughout the imaging process.

At step 370, the device’s SDK 230 or API returns errors or throws exceptions during the image acquisition operations performed in step 360. For example, the return value from the device SDK 230 or API may indicate whether there is an explicit exception thrown or whether there is an error code in the return value. The software/plugin 380 may also determine whether the return value matches what the system expects (e.g., is it an appropriate context for the returned value?). During step 370 one or more of the monitoring modules may evaluate the device responses 307 received from the imaging device 105 to identify error conditions, performance anomalies, or operational failures that occur during imaging procedures. In some cases, communication timeouts, image quality issues, calibration failures, or hardware malfunctions may be analyzed that could affect imaging results or device functionality. The severity and impact of detected errors may be assessed to determine appropriate logging and notification procedures. Error conditions may be classified according to predefined categories that support automated troubleshooting and maintenance recommendations.

When the result of step 370 is a detected error or exception during image acquisition, the method 300 proceeds to a step 380 where comprehensive error information is logged to the backend server 109. The step 380 may capture detailed error descriptions, exception stack traces, and contextual information that supports diagnostic and troubleshooting activities. In some cases, the step 380 may record the specific error or status code retrieved from the device's SDK 230 or API, along with error type classifications and criticality assessments. The step 380 may generate detailed hints or instructions for resolving the detected errors based on the error characteristics and device specifications. The step 380 may compile the status data 108 that includes device brand, model, serial number, PC name, practice ID, organization ID, user ID, status type, error type, error text, exception details, error hints, and software version information to provide comprehensive error documentation.

When the step 370 determination results in no errors occurred during image acquisition, the method 300 proceeds to a step 390 where successful operation status is logged to the backend server 109. The step 390 may record that the imaging operations completed successfully without errors or performance issues, along with comprehensive operational context information. In some cases, the step 390 may capture performance metrics, timing information, and operational parameters that document successful device utilization. The step 390 may generate the status data 108 that includes device identification information, user details, location data, and operational context that supports performance tracking and maintenance planning. The step 390 may record status type indicators that classify the successful operation and maintain historical records of device performance for trend analysis and predictive maintenance applications.

### Example Date Handling

With reference back to FIG. 1, the database system 113 may implement relational database management systems that organize the processed data 114 into structured tables with defined relationships, data types, and integrity constraints that ensure consistent data organization and retrieval performance. The database system 113 may also support non-relational database architectures including document databases, key-value stores, or graph databases that accommodate varying data structures and access patterns associated with different device types and monitoring requirements. In some cases, the database system 113 may utilize hybrid storage approaches that combine relational and non-relational technologies to optimize performance for different data categories, query patterns, or analytical workloads. The database system 113 may incorporate data partitioning strategies that distribute large datasets across multiple storage volumes or geographic locations to enhance query performance and support scalable growth as device monitoring operations expand.

The database system 113 may maintain historical data for long-term analysis of device health and performance trends through comprehensive data retention policies that preserve operational records across extended time periods. Historical data storage may include device status information, error logs, performance metrics, usage patterns, and environmental context data that enable trend analysis, comparative studies, and predictive maintenance applications. In some cases, the database system 113 may implement data archiving procedures that migrate older records to cost-effective storage tiers while maintaining accessibility for analytical queries and reporting functions. The database system 113 may support data compression techniques, indexing strategies, and query optimization methods that enable efficient access to historical information despite large data volumes accumulated over months or years of device monitoring operations.

As further shown in FIG. 1, the database system 113 may interface with analytics tools through application programming interfaces that enable external systems to access stored device information for visualization, analysis, and reporting purposes. The database system 113 may provide web-based APIs that support RESTful communication protocols, enabling analytics dashboards, business intelligence tools, and custom applications to retrieve device data through standardized query interfaces. In some cases, the database system 113 may support direct database replication mechanisms that synchronize device monitoring data with external analytics platforms, data warehouses, or business intelligence systems through automated data transfer processes. The database system 113 may enable standard database queries using Structured Query Language or other query languages that allow analytics tools to access device information through native database connections and established query protocols.

Referring back to FIG. 2, the backend server 206 may process the status data 214 received from the device health monitoring plugin 202 and generate processed data 216 that conforms to standardized storage formats compatible with a database system 207. The database system 207 may implement similar storage capabilities as the database system 113, providing both local and cloud-hosted deployment options that accommodate different organizational preferences and infrastructure constraints. In some cases, the database system 207 may support distributed database architectures that enable multiple backend servers to contribute device monitoring data to a centralized storage system, facilitating large-scale monitoring operations across multiple practice locations or organizational units. The database system 207 may incorporate data synchronization mechanisms that ensure consistency across distributed storage nodes while maintaining high availability and fault tolerance capabilities.

The database system 207 may provide equivalent interface to tools suited for analytics and interactive visualization through various connectivity options that enable external systems to access stored device monitoring information. The database system 207 may support direct database connections that allow analytics platforms to execute queries against stored device data using standard database protocols and query languages. In some cases, the database system 207 may implement data export functions that generate formatted data files compatible with specific analytics tools, visualization platforms, or business intelligence applications. The database system 207 may provide real-time data streaming capabilities that enable analytics tools to receive continuous updates of device status information as monitoring events occur, supporting dynamic dashboards and real-time alerting systems.

In some embodiments, the database system 207 may implement data quality management procedures that validate, cleanse, and standardize device monitoring information before storage to ensure consistency and reliability of analytical results. The database system 207 may support metadata management capabilities that document data lineage, transformation procedures, and quality metrics associated with stored device information. In some cases, the database system 207 may provide data governance features that control access permissions, audit data usage, and maintain compliance with regulatory requirements or organizational policies governing device monitoring information. The database system 207 may incorporate backup and recovery mechanisms that protect stored device data against hardware failures, data corruption, or other operational disruptions while maintaining continuous availability for monitoring and analytics operations.

### Example Reporting

Referring back to FIG. 1, the user interface 110 may function as a reporting module that provides comprehensive insights into the operational health of dental imaging devices through various presentation formats and analytical capabilities. The user interface 110 may generate detailed reports that document device status information, error history patterns, and user interaction data collected through the monitoring operations performed by the device interface 102. In some cases, the user interface 110 may support multiple stakeholder access levels including practice administrators, IT personnel, device technicians, and clinical staff who require different types of operational information and analytical perspectives. The user interface 110 may provide real-time dashboards that display current device status across multiple locations, along with historical trend analysis that identifies patterns in device performance over extended time periods.

The user interface 110 may incorporate various interface methods that accommodate different stakeholder preferences and technical capabilities for accessing device monitoring information. Web-based dashboard interfaces may provide interactive visualizations that enable stakeholders to explore device performance data through graphical charts, statistical summaries, and customizable filtering options that focus analysis on specific time periods, device types, or operational conditions. In some cases, the user interface 110 may support mobile application interfaces that enable remote access to device status information through smartphones or tablet devices, allowing IT personnel and practice managers to monitor device health while away from practice locations. The user interface 110 may also provide application programming interface access that enables custom software applications or third-party systems to retrieve device monitoring data through programmatic interfaces that support automated reporting and integration workflows.

In some embodiments, the user interface 110 may generate an interface request 112 that specifies the particular analytical parameters, data filtering criteria, and report formatting requirements needed to produce stakeholder-specific insights into device operational health. The interface request 112 may contain query specifications that define the scope of data analysis operations, including device identification filters, time range parameters, error condition categories, or performance metric thresholds that focus the reporting on specific operational aspects. In some cases, the interface request 112 may specify aggregation functions that calculate summary statistics across multiple devices, comparative analyses between different practice locations, or trend calculations that identify performance changes over time. The interface request 112 may also define presentation formatting parameters that determine how the analytical results are structured and displayed within the user interface 110 to meet the specific information needs of different stakeholder groups.

The backend server 109 may process the interface request 112 by executing analytical queries against the processed data 114 stored within the database system 113, applying statistical calculations and data transformation operations that generate comprehensive insights into device operational patterns. The backend server 109 may implement analytical algorithms that identify error frequency trends, device utilization patterns, performance degradation indicators, or maintenance scheduling recommendations based on the historical operational data collected through the monitoring system. In some cases, the backend server 109 may correlate device performance information with environmental factors such as practice location characteristics, user behavior patterns, or operational workflow variations that influence device health and performance outcomes. The backend server 109 may generate standardized data 111 that contains the analytical results formatted according to the presentation requirements specified within the interface request 112.

In some embodiments, the standardized data 111 may contain comprehensive reporting information that provides stakeholders with actionable insights into device status conditions, error history analysis, and user interaction patterns that support operational decision-making and maintenance planning activities. The standardized data 111 may include device status summaries that document the current operational state of monitored devices, along with historical status trends that identify patterns in device availability, performance consistency, or operational reliability across different time periods. In some cases, the standardized data 111 may contain error history reports that categorize detected issues according to error type classifications, frequency of occurrence, resolution status, or impact severity levels that enable prioritized troubleshooting and maintenance activities. The standardized data 111 may also include user interaction analytics that document device usage patterns, operator performance metrics, or training effectiveness indicators that support workflow optimization and staff development initiatives.

The user interface 110 may support connectivity to analytics dashboards that provide advanced visualization capabilities for exploring device monitoring data through interactive charts, statistical analyses, and customizable reporting formats. Analytics dashboard integration may enable stakeholders to create personalized views of device performance information, configure automated alerting systems that notify personnel of operational issues, or generate scheduled reports that document device health trends for management review purposes. In some cases, the user interface 110 may interface with business intelligence tools that combine device monitoring data with other practice management information to provide comprehensive operational insights that support strategic planning and resource allocation decisions. The user interface 110 may also connect to specialized analytics platforms that focus on predictive maintenance applications, enabling advanced statistical modeling and forecasting capabilities that anticipate device maintenance requirements or replacement planning needs.

Referring back to FIG. 2, an interface module 205 may provide equivalent reporting and analytical capabilities within the plugin-based monitoring architecture, enabling stakeholders to access comprehensive device health insights through various presentation methods and analytical tools. The interface module 205 may support similar dashboard interfaces, mobile applications, and programmatic access methods as described for the user interface 110, while accommodating the specific data formats and communication protocols associated with the device health monitoring plugin 202 implementation. In some cases, the interface module 205 may provide enhanced integration capabilities that leverage the plugin architecture to access additional device information or operational context that may not be available through standard device interface methods. The interface module 205 may generate interface requests 217 that specify analytical parameters and reporting requirements for processing by the backend server 206.

The backend server 206 may process the interface requests 217 through analytical operations that examine the processed data 216 stored within the database system 207, generating comprehensive insights into device operational health and performance characteristics. The backend server 206 may implement equivalent analytical capabilities as described for the backend server 109, including trend analysis, error pattern recognition, performance monitoring, and predictive maintenance recommendations based on the device monitoring data collected through the device health monitoring plugin 202. In some cases, the backend server 206 may provide enhanced analytical features that utilize the additional contextual information captured through the plugin interception mechanisms to generate more detailed insights into device operational patterns and user interaction behaviors. The backend server 206 may return standardized data 215 that contains the analytical results formatted according to the requirements specified within the interface requests 217.

In some embodiments, the interface module 205 may support connectivity to external analytics systems through web APIs that enable programmatic access to device monitoring data for integration with third-party analytical platforms, business intelligence tools, or custom reporting applications. Web API connectivity may provide RESTful interfaces that support standard HTTP communication protocols, enabling external systems to retrieve device status information, error history data, or performance metrics through authenticated API calls that maintain data security and access control requirements. In some cases, the interface module 205 may support direct database replication mechanisms that synchronize device monitoring data with external analytics platforms through automated data transfer processes that maintain real-time or near-real-time data availability for analytical operations. The interface module 205 may also enable standard database queries through SQL interfaces or other query languages that allow analytics tools to access device information through native database connections and established query protocols.

The database system 207 may provide comprehensive data access capabilities that support various analytical tools and reporting platforms through multiple connectivity options that accommodate different technical requirements and integration preferences. The database system 207 may implement web-based APIs that enable external analytics dashboards to retrieve device monitoring data through standardized communication protocols, supporting real-time data visualization and interactive reporting capabilities. In some cases, the database system 207 may support direct database replication services that automatically synchronize device monitoring information with external data warehouses, business intelligence platforms, or specialized analytics systems that require local data access for performance optimization or regulatory compliance purposes. The database system 207 may also provide standard database query interfaces that enable analytics tools to execute SQL queries or other database operations directly against the stored device monitoring data, supporting custom reporting applications and specialized analytical workflows that require flexible data access capabilities.

The troubleshooting module may provide comprehensive diagnostic capabilities that enable stakeholders to identify and resolve device operational issues through systematic analysis of the logged data collected during device monitoring operations. The troubleshooting module may process error codes, status information, and contextual data to generate detailed explanations of detected problems along with specific guidance for addressing identified issues. In some cases, the troubleshooting module may correlate current error conditions with historical patterns stored in the database system to identify recurring problems and recommend preventive measures that reduce the likelihood of future occurrences. The troubleshooting module may support various stakeholder skill levels by providing both technical diagnostic information for experienced personnel and simplified explanations for users who may lack specialized knowledge of dental imaging device operations.

The troubleshooting module may generate detailed error context information that explains the meaning and implications of specific error codes or status conditions detected during device operations. Error context generation may involve analyzing the device brand, model, and operational environment to provide device-specific explanations that account for manufacturer-specific behaviors and known operational characteristics. In some cases, the troubleshooting module may access comprehensive error databases that contain detailed descriptions of error conditions across multiple device types, enabling accurate interpretation of error codes that may vary between different manufacturers or device models. The troubleshooting module may also incorporate environmental context information such as PC specifications, software versions, and network conditions to provide comprehensive diagnostic assessments that consider all factors that may contribute to observed operational issues.

The troubleshooting module may provide guidance for resolving known errors through structured resolution procedures that guide stakeholders through systematic diagnostic and repair processes. Resolution guidance may include step-by-step instructions that address common causes of detected error conditions, along with verification procedures that confirm successful resolution of identified problems. In some cases, the troubleshooting module may recommend specific maintenance actions such as device calibration procedures, software updates, driver reinstallation, or hardware inspection activities based on the characteristics of detected errors and the operational history of affected devices. The troubleshooting module may also provide escalation procedures that direct stakeholders to appropriate technical support resources when automated resolution guidance proves insufficient for addressing complex or unusual error conditions.

The troubleshooting module may implement automated suggestion capabilities that analyze logged data patterns to identify optimal resolution strategies for common device issues without requiring manual diagnostic procedures. Automated suggestion generation may utilize pattern recognition algorithms that examine historical error resolution data to identify successful troubleshooting approaches for similar error conditions and device configurations. In some cases, the troubleshooting module may correlate current error symptoms with previously resolved issues to recommend proven solution strategies that have demonstrated effectiveness in comparable operational environments. The automated suggestion system may also consider device age, usage patterns, and maintenance history when generating recommendations to ensure that suggested solutions account for device-specific factors that may influence resolution effectiveness.

The troubleshooting module may provide solutions for resolving common device issues through comprehensive knowledge bases that document proven resolution procedures for frequently encountered operational problems. Solution databases may contain detailed troubleshooting workflows that address communication failures, calibration errors, image quality issues, and hardware malfunctions across various device types and operational configurations. In some cases, the troubleshooting module may maintain manufacturer-specific solution libraries that provide device-specific resolution procedures tailored to the operational characteristics and known issues associated with particular device brands and models. The troubleshooting module may also incorporate user-contributed solutions and field experience data that expand the available resolution options beyond manufacturer-provided documentation.

The troubleshooting module may analyze logged data to identify trends and patterns that indicate emerging device issues before complete operational failures occur, enabling proactive maintenance interventions that prevent service disruptions. Trend analysis capabilities may examine error frequency patterns, performance degradation indicators, and usage anomalies to detect early warning signs of potential device problems. In some cases, the troubleshooting module may correlate multiple data sources including error logs, performance metrics, and environmental conditions to identify complex failure patterns that may not be apparent through individual data analysis. The troubleshooting module may generate predictive maintenance recommendations that specify optimal timing for preventive actions such as device cleaning, calibration, component replacement, or software updates based on observed operational trends and historical maintenance effectiveness data.

The troubleshooting module may provide insights into device health through comprehensive analytical capabilities that evaluate operational performance across multiple dimensions including reliability, efficiency, and user satisfaction metrics. Device health assessment may incorporate statistical analysis of error rates, operational success percentages, and performance consistency measurements to generate overall health scores that enable comparative evaluation across multiple devices and practice locations. In some cases, the troubleshooting module may identify correlations between device health indicators and external factors such as environmental conditions, usage intensity, or operator behavior patterns that influence device performance outcomes. The troubleshooting module may also generate health trend projections that estimate future device performance based on current operational patterns and historical degradation rates.

The troubleshooting module may recommend maintenance actions through systematic analysis of device operational data, error patterns, and performance trends that indicate optimal timing and scope for preventive maintenance activities. Maintenance recommendation algorithms may consider device age, usage intensity, error frequency, and performance metrics to generate customized maintenance schedules that balance operational availability with preventive care requirements. In some cases, the troubleshooting module may recommend specific maintenance procedures such as sensor cleaning, calibration verification, software updates, or component inspections based on the operational characteristics and known maintenance requirements of particular device types. The troubleshooting module may also provide maintenance prioritization guidance that helps stakeholders allocate limited maintenance resources to devices that demonstrate the greatest need for preventive attention based on health assessment results and operational importance factors.

### Example Notification System

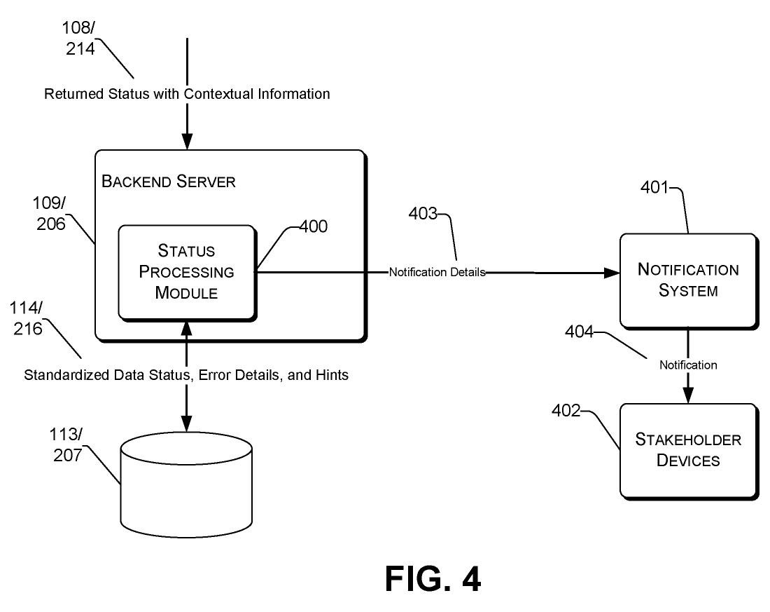

Referring to FIG. 4, the system may incorporate comprehensive notification capabilities that enable real-time communication with stakeholders when device operational issues or status changes occur during monitoring activities. A status processing module 400 operates within the backend server architecture to analyze incoming device status information and determine when notification events should be triggered based on predefined operational criteria. The status processing module 400 may receive the status data containing device operational information, error conditions, and contextual parameters collected through the monitoring system, and may process this information through rule-based evaluation algorithms that assess the significance and urgency of detected conditions. In some cases, the status processing module 400 may implement configurable threshold parameters that define the operational conditions, error severity levels, or performance anomalies that warrant stakeholder notification, enabling customized alerting behavior that aligns with organizational preferences and operational priorities.

The status processing module 400 may evaluate device status information through systematic analysis procedures that categorize detected conditions according to predefined notification criteria including error type classifications, device criticality levels, and operational impact assessments. The status processing module 400 may implement rule-based decision algorithms that examine multiple data elements including error codes, device identification information, location parameters, and historical context to determine appropriate notification actions for detected conditions. In some cases, the status processing module 400 may correlate current device status with historical patterns to identify recurring issues, escalating problems, or unusual operational anomalies that may warrant immediate stakeholder attention. The status processing module 400 may also consider time-based factors such as business hours, maintenance schedules, or operational priorities when determining notification timing and recipient selection for detected device conditions.

With continued reference to FIG. 4, the status processing module 400 may generate notification details 403 that contain comprehensive information about detected device conditions formatted for transmission through various communication channels to appropriate stakeholders. The notification details 403 may include device identification information such as brand, model, and serial number specifications that enable stakeholders to identify the specific equipment experiencing operational issues. In some cases, the notification details 403 may contain location information including practice identification, organizational context, and geographic details that enable stakeholders to understand the operational environment and potential impact of detected conditions. The notification details 403 may also incorporate error descriptions, severity assessments, and recommended response actions that provide stakeholders with actionable information for addressing detected device issues without requiring additional diagnostic procedures.

The status processing module 400 may format the notification details 403 according to the communication requirements and technical capabilities of different notification delivery methods including text-based messaging, email communication, and application-based push notification systems. Text-based notification formatting may involve generating concise message content that conveys essential device status information within the character limitations and formatting constraints associated with SMS messaging systems. In some cases, email notification formatting may enable more comprehensive information presentation including detailed error descriptions, historical context, and embedded links to analytical dashboards or troubleshooting resources that support stakeholder response activities. Application-based push notification formatting may accommodate interactive message elements that enable stakeholders to acknowledge receipt, initiate response actions, or access additional device information through mobile application interfaces.

As shown in FIG. 4, a notification system 401 receives the notification details 403 from the status processing module 400 and manages the distribution of alert messages to appropriate stakeholder communication devices through various delivery channels and communication protocols. The notification system 401 may comprise commercially available communication platforms such as Twilio or SendGrid that provide comprehensive messaging services including SMS delivery, email transmission, and push notification capabilities through established communication infrastructure. In some cases, the notification system 401 may implement custom communication solutions that integrate with organizational communication systems, internal messaging platforms, or specialized alerting infrastructure that aligns with existing operational procedures and security requirements. The notification system 401 may support multiple communication protocols simultaneously, enabling redundant message delivery through different channels to ensure reliable stakeholder notification even when individual communication methods experience service disruptions or delivery failures.

The notification system 401 may implement delivery management capabilities that track message transmission status, confirm successful delivery to target devices, and provide delivery failure notifications when communication attempts are unsuccessful. Delivery tracking functionality may monitor message status through various stages including transmission initiation, carrier acceptance, device delivery, and recipient acknowledgment to provide comprehensive visibility into notification effectiveness. In some cases, the notification system 401 may implement retry mechanisms that attempt alternative delivery methods when primary communication channels fail, ensuring that stakeholders receive device status information despite temporary communication disruptions or device availability issues. The notification system 401 may also provide delivery analytics that document notification frequency, response times, and communication effectiveness metrics that support optimization of alerting procedures and stakeholder communication strategies.

In some embodiments, the notification system 401 may distribute notification messages 404 to stakeholder devices 402 through multiple communication channels that accommodate different stakeholder preferences, technical capabilities, and operational requirements. The notification messages 404 may contain all or selected portions of the device status information collected through the monitoring system, formatted according to the communication channel capabilities and stakeholder information needs. In some cases, the notification messages 404 may include abbreviated status summaries for SMS delivery, comprehensive error reports for email communication, or interactive alert elements for application-based push notifications that enable stakeholders to access additional device information or initiate response actions directly from the notification interface. The notification messages 404 may also incorporate priority indicators, urgency classifications, or escalation procedures that guide stakeholder response activities based on the severity and operational impact of detected device conditions.

The stakeholder devices 402 may include various communication endpoints including smartphones, personal computers, tablet devices, and other communication-capable equipment that enable stakeholders to receive and respond to device status notifications regardless of their physical location or operational context. Smartphone integration may enable stakeholders to receive SMS messages, email notifications, and application-based push alerts through cellular communication networks, wireless internet connections, or mobile data services that provide continuous connectivity for real-time notification delivery. In some cases, personal computer integration may support email notification delivery, desktop application alerts, or web-based notification interfaces that enable stakeholders to access comprehensive device information and analytical resources through full-featured computing environments. Tablet device integration may provide hybrid capabilities that combine mobile accessibility with enhanced display capabilities for reviewing detailed device status information and accessing troubleshooting resources while maintaining operational mobility.

The stakeholder devices 402 may support various notification presentation methods that accommodate different user preferences and operational contexts including visual alerts, audio notifications, and vibration indicators that ensure stakeholder awareness of device status changes. Visual notification presentation may include text message displays, email content rendering, or application-based alert interfaces that provide comprehensive information presentation through graphical user interfaces. In some cases, audio notification capabilities may generate distinctive alert tones, spoken message content, or customizable sound patterns that enable stakeholder awareness of device status changes even when visual attention is directed toward other activities. Vibration notification functionality may provide tactile alerts that ensure stakeholder awareness of device status messages in environments where audio or visual notifications may not be practical or appropriate.

In some embodiments, the notification system 401 may implement configurable rule-based processing that enables organizations to customize notification behavior according to operational priorities, stakeholder responsibilities, and device criticality assessments. Rule configuration capabilities may enable administrators to define specific conditions that trigger notification events including error type categories, device location parameters, time-based criteria, or performance threshold violations that warrant stakeholder attention. In some cases, rule-based processing may support escalation procedures that automatically notify additional stakeholders or management personnel when initial notifications are not acknowledged within specified time periods, ensuring that device issues receive appropriate attention despite stakeholder availability limitations. The notification system 401 may also implement filtering capabilities that prevent notification flooding by consolidating multiple related alerts, suppressing duplicate messages, or implementing notification frequency limits that balance stakeholder awareness with communication efficiency.

The status processing module 400 may provide real-time alert generation capabilities that enable immediate stakeholder notification when critical errors are detected during device monitoring operations, supporting rapid response to operational issues that may impact clinical activities or patient care procedures. Real-time processing may involve continuous monitoring of incoming status data with immediate evaluation against notification criteria to minimize the time delay between error detection and stakeholder notification. In some cases, real-time alert generation may prioritize critical error conditions such as complete device failures, safety-related malfunctions, or operational disruptions that require immediate attention over less urgent status changes or routine operational events. The status processing module 400 may implement priority-based notification queuing that ensures critical alerts receive immediate processing and delivery while managing routine notifications through standard processing procedures that balance system performance with comprehensive monitoring coverage.

The dental imaging device health monitoring system may incorporate comprehensive machine learning components that analyze historical operational data to develop predictive models for anticipating device failures and performance degradations before operational disruptions occur. Machine learning algorithms may process extensive datasets containing device performance metrics, error logs, usage patterns, and environmental factors collected through continuous monitoring operations across multiple practice locations and device types. In some cases, the machine learning components may examine correlations between device operational characteristics and failure patterns to identify predictive indicators that enable proactive maintenance interventions. The machine learning system may utilize diverse data sources including device-specific parameters such as brand specifications, model characteristics, and serial number tracking, along with environmental context information including practice location details, user behavior patterns, and operational workflow variations that influence device performance outcomes.

The machine learning components may implement supervised learning models that utilize labeled training datasets containing historical device operational records paired with known outcomes such as device failures, maintenance events, or performance degradation incidents. Supervised learning algorithms may include decision tree implementations that analyze device operational parameters through hierarchical decision structures to classify devices according to failure risk categories or maintenance priority levels. In some cases, neural network architectures may process complex patterns within historical device data through interconnected processing layers that identify non-linear relationships between operational variables and device health outcomes. The supervised learning models may incorporate multiple input variables including error frequency patterns, performance consistency measurements, usage intensity metrics, and environmental condition data to generate comprehensive risk assessments for individual devices or device populations across practice networks.

The machine learning system may utilize cross-validation techniques during model training procedures to ensure that predictive algorithms generalize effectively across different device types, operational environments, and practice configurations without overfitting to specific training datasets. Cross-validation procedures may involve partitioning historical device data into training and validation subsets that enable iterative model refinement through systematic testing of predictive accuracy across diverse operational scenarios. In some cases, k-fold cross-validation methods may divide available training data into multiple segments that enable comprehensive evaluation of model performance across different data distributions and operational contexts. The cross-validation process may assess model accuracy through statistical metrics including precision, recall, and F1-score measurements that quantify the effectiveness of failure prediction algorithms across various device categories and operational conditions.

Machine learning models may undergo continuous updating procedures through adaptive learning processes that incorporate new operational data as monitoring activities generate additional device performance information over time. Adaptive learning mechanisms may automatically retrain predictive algorithms when sufficient new data becomes available, enabling model refinement that accounts for evolving device characteristics, changing operational patterns, or emerging failure modes that may not have been present in original training datasets. In some cases, incremental learning approaches may update model parameters through online learning algorithms that process new device data in real-time without requiring complete model retraining procedures. The adaptive learning system may implement change detection algorithms that identify when device operational patterns deviate significantly from historical norms, triggering model updates that maintain predictive accuracy despite evolving operational conditions or device aging effects.

The machine learning components may analyze usage patterns through statistical analysis of device operational frequency, utilization intensity, and workflow integration characteristics that influence device wear patterns and maintenance requirements. Usage pattern analysis may examine temporal variations in device activity including daily usage cycles, weekly operational patterns, and seasonal variations that affect device stress levels and component degradation rates. In some cases, the machine learning system may correlate usage intensity measurements with device performance metrics to identify optimal operational thresholds that balance clinical productivity with device longevity considerations. The usage pattern analysis may also evaluate operator behavior characteristics including device handling procedures, operational technique variations, and maintenance compliance patterns that influence device reliability and performance consistency across different practice environments.

Predictive maintenance recommendations may be generated through machine learning algorithms that analyze device performance trends, usage patterns, and historical maintenance effectiveness data to determine optimal timing and scope for preventive maintenance activities. The machine learning system may process device health indicators including error rate trends, performance degradation measurements, and component aging assessments to predict when maintenance interventions will provide maximum benefit for device reliability and operational availability. In some cases, the predictive algorithms may recommend specific maintenance procedures such as calibration verification, component cleaning, software updates, or hardware inspections based on device-specific characteristics and observed performance patterns. The machine learning system may also generate recommendations for device recalibration activities by analyzing measurement accuracy trends, calibration drift patterns, and environmental factors that affect device precision over time.

The machine learning components may provide recommendations for part replacement activities through analysis of component failure patterns, device age characteristics, and operational stress factors that influence component lifespan and replacement timing. Part replacement recommendations may consider device usage intensity, environmental conditions, and historical component reliability data to predict when specific components are likely to fail or degrade beyond acceptable performance thresholds. In some cases, the machine learning system may analyze warranty information, replacement cost factors, and operational impact assessments to optimize part replacement timing that balances maintenance costs with operational reliability requirements. The predictive algorithms may also consider component availability, maintenance scheduling constraints, and practice operational priorities when generating part replacement recommendations that minimize service disruptions while maintaining device performance standards.

Machine learning models may incorporate feature extraction methods that identify the most predictive indicators within complex device operational datasets, enabling focused analysis of data elements that provide the greatest value for failure prediction and maintenance planning applications. Feature extraction algorithms may analyze correlation patterns between different operational variables to identify combinations of metrics that provide enhanced predictive capability compared to individual data elements. In some cases, dimensionality reduction techniques may process high-dimensional device data through principal component analysis or other mathematical transformations that identify underlying patterns while reducing computational complexity and improving model training efficiency. The feature extraction process may also incorporate domain expertise regarding device operational characteristics and failure mechanisms to ensure that machine learning models focus on technically relevant data patterns that align with established understanding of device behavior and maintenance requirements.

The machine learning system may implement ensemble methods that combine multiple predictive algorithms to generate more robust and accurate failure predictions than individual models operating independently. Ensemble approaches may utilize voting mechanisms that aggregate predictions from multiple machine learning models including decision trees, neural networks, and statistical regression algorithms to generate consensus predictions with improved reliability and reduced prediction variance. In some cases, boosting techniques may sequentially train multiple models that focus on correcting prediction errors from previous algorithms, resulting in ensemble systems with enhanced accuracy for challenging prediction scenarios. The ensemble methods may also incorporate model weighting strategies that assign greater influence to algorithms that demonstrate superior performance for specific device types or operational conditions, enabling customized prediction approaches that account for the diverse characteristics of different dental imaging equipment categories.

The dental imaging device health monitoring system may implement comprehensive scalability features that enable deployment across enterprise-level organizations with thousands of devices distributed across multiple geographic locations and practice sites. Scalable architecture design may accommodate varying organizational sizes from individual dental practices to large dental support organizations that operate extensive networks of clinical facilities. In some cases, the system may support horizontal scaling approaches that distribute monitoring workloads across multiple processing nodes, enabling performance optimization as device populations grow and monitoring data volumes increase over time. The scalable architecture may incorporate load balancing mechanisms that distribute device communication requests and data processing operations across available system resources to maintain consistent response times and operational reliability despite varying usage patterns and peak demand periods.

Multi-tenant architecture capabilities may enable the system to serve multiple organizations simultaneously while maintaining data isolation and security boundaries between different customer environments. Each organization may operate within dedicated logical partitions that provide isolated access to device monitoring data, configuration settings, and analytical resources without interference from other organizational deployments. In some cases, multi-tenant implementations may utilize shared infrastructure resources including processing capacity, storage systems, and network connectivity while maintaining strict data segregation through access control mechanisms and encryption protocols. The multi-tenant architecture may support customizable configuration options that enable each organization to define monitoring parameters, notification preferences, and reporting formats according to organizational policies and operational requirements without affecting other tenant environments.

The system may incorporate distributed database architectures that enable data aggregation from multiple practice locations into centralized storage systems that support comprehensive analysis and reporting across entire organizational networks. Centralized data aggregation may involve collecting device monitoring information from individual practice sites through secure communication channels and consolidating this information within central database systems that provide unified access to organizational device health data. In some cases, the centralized database architecture may implement data partitioning strategies that organize device information according to geographic regions, practice types, or organizational divisions while maintaining global accessibility for enterprise-level analysis and reporting functions. The data aggregation process may support real-time synchronization mechanisms that ensure central databases contain current device status information despite network latency or temporary communication disruptions between practice locations and central processing systems.

The system may support broader scalable platform implementations where each organization operates dedicated backend server infrastructure and database systems that synchronize with consolidated data sources through various integration methods. Individual organizational deployments may maintain local processing capabilities and data storage systems that provide operational independence while participating in larger data aggregation networks that enable comparative analysis and benchmarking across multiple organizations. In some cases, the scalable platform architecture may utilize data warehouse integration methods that extract device monitoring information from individual organizational databases and transform this data into standardized formats suitable for consolidated analysis and reporting applications. The platform may also implement data lake integration approaches that accommodate diverse data formats and structures from different organizational deployments while providing unified access to aggregated device monitoring information.

Data warehouse integration methods may enable systematic extraction, transformation, and loading of device monitoring data from distributed organizational databases into centralized analytical systems that support enterprise-level reporting and business intelligence applications. The data warehouse architecture may implement dimensional modeling approaches that organize device monitoring information according to analytical dimensions including time periods, device categories, geographic locations, and organizational hierarchies that facilitate comprehensive trend analysis and comparative studies. In some cases, the data warehouse may support historical data retention policies that preserve device monitoring information across extended time periods while implementing data archiving strategies that optimize storage costs and query performance for different analytical workloads. The integration process may utilize automated data pipeline mechanisms that schedule regular data extraction and transformation operations while monitoring data quality and consistency across multiple source systems.

Data lake integration capabilities may provide flexible storage architectures that accommodate diverse data types and formats generated by different device monitoring implementations while maintaining unified access to consolidated information through standardized query interfaces. The data lake architecture may support both structured data from relational database systems and unstructured data including device logs, diagnostic reports, and multimedia content that provide comprehensive operational context for analytical applications. In some cases, the data lake may implement schema-on-read approaches that enable flexible data analysis without requiring predefined data structures, allowing analytical applications to process device monitoring information according to specific analytical requirements and emerging use cases. The data lake integration may also support real-time data streaming capabilities that enable continuous ingestion of device monitoring information from distributed organizational deployments while maintaining data consistency and availability for analytical operations.

Fabric integration methods may provide comprehensive data integration platforms that combine multiple data sources, processing capabilities, and analytical tools within unified environments that support complex analytical workflows and collaborative analysis activities. The fabric architecture may integrate device monitoring data with other organizational information systems including practice management platforms, financial systems, and operational databases to provide comprehensive business intelligence capabilities that support strategic planning and operational optimization decisions. In some cases, the fabric integration may implement microservices architectures that decompose data processing and analytical functions into modular components that can be scaled independently according to specific workload requirements and performance objectives. The fabric platform may also provide collaborative analytical environments that enable multiple stakeholders to access and analyze consolidated device monitoring information through shared dashboards, reporting tools, and analytical applications.

The scalable architecture may implement modular design principles that enable the addition of new device types and enhanced functionality without requiring modifications to existing system components or disrupting ongoing monitoring operations. Modular architecture design may utilize standardized interfaces and communication protocols that enable new device integration modules to connect with existing monitoring infrastructure through well-defined application programming interfaces and data exchange formats. In some cases, the modular approach may support plugin architectures that enable third-party developers to create specialized monitoring components for emerging device technologies or proprietary equipment that may not conform to standard integration methods. The modular design may also facilitate feature expansion through independent development and deployment of new analytical capabilities, reporting functions, or integration options that enhance system functionality while maintaining backward compatibility with existing deployments.

Support for emerging technologies in dental imaging may be provided through flexible integration frameworks that accommodate evolving device communication protocols, data formats, and operational characteristics associated with advancing dental imaging equipment. The system architecture may implement abstraction layers that isolate device-specific communication details from core monitoring functions, enabling rapid adaptation to new device technologies without requiring extensive system modifications. In some cases, the emerging technology support may include artificial intelligence integration capabilities that enable advanced analytical functions including image analysis, automated diagnostic assistance, or predictive maintenance algorithms that leverage machine learning technologies. The system may also provide integration capabilities for cloud-based device services, Internet of Things connectivity, and mobile device interfaces that reflect evolving trends in dental imaging technology and practice management workflows.

Unified management capabilities may enable centralized administration of distributed device monitoring operations through comprehensive management interfaces that provide visibility and control over thousands of devices across multiple practice locations. The unified management system may support hierarchical organizational structures that enable different levels of administrative access and control according to organizational roles and responsibilities. In some cases, the management interface may provide centralized configuration capabilities that enable administrators to define monitoring parameters, notification settings, and reporting preferences that apply across multiple practice locations while allowing local customization for site-specific requirements. The unified management system may also implement centralized user management functions that control access permissions, authentication procedures, and audit logging across distributed monitoring deployments while maintaining security and compliance requirements.

Analysis capabilities within the scalable architecture may support comprehensive analytical workflows that process device monitoring data from thousands of devices to generate organizational insights, performance benchmarks, and operational optimization recommendations. The analytical system may implement distributed processing architectures that utilize parallel computing resources to analyze large datasets efficiently while maintaining interactive response times for dashboard interfaces and reporting applications. In some cases, the analysis capabilities may include statistical modeling functions that identify patterns and trends across device populations, enabling comparative analysis between different practice locations, device types, or operational configurations. The analytical system may also provide predictive modeling capabilities that utilize machine learning algorithms to forecast device maintenance requirements, replacement planning needs, or operational optimization opportunities based on aggregated data from multiple organizational deployments.

The dental imaging device health monitoring system operates through comprehensive integration of multiple components that work together to provide continuous oversight of device operational status and performance characteristics. The system establishes communication pathways between dental imaging equipment and monitoring infrastructure through various integration methods including native software development kits, TWAIN interfaces, DirectShow protocols, and Universal Video Class implementations that accommodate diverse device types and manufacturer specifications. Data collection processes capture device status information, error conditions, and operational context through systematic monitoring procedures that occur during normal imaging workflows without disrupting clinical activities or established practice procedures. The collected information flows through standardized processing pipelines that transform raw device data into structured formats suitable for storage, analysis, and reporting applications across different organizational environments and deployment scenarios.

Operational workflows begin when dental imaging software initiates communication with connected devices through established application programming interfaces that enable device control and status monitoring functions. The monitoring system intercepts or captures device communication activities to extract operational information including status codes, error conditions, performance metrics, and contextual data that provide comprehensive visibility into device health and operational characteristics. Device responses containing operational results, diagnostic information, and status indicators are processed through monitoring modules that evaluate the significance of detected conditions and determine appropriate logging and notification actions based on predefined criteria and organizational policies. The monitoring process continues throughout imaging procedures to capture comprehensive operational data that documents device performance across various clinical scenarios and usage patterns.

Data processing operations transform captured device information through standardization procedures that normalize data formats, validate information integrity, and enrich operational records with additional contextual details including user identification, location information, and environmental parameters. The processing system correlates device-specific information with organizational databases that provide practice identification, equipment inventories, and administrative context that enable comprehensive tracking and analysis across multiple locations and device populations. Standardized data records are generated through transformation algorithms that structure device information according to consistent schemas that support efficient storage, retrieval, and analysis operations regardless of the original device communication protocols or data formats. The processed information is transmitted to storage systems through secure communication channels that maintain data integrity and confidentiality while enabling distributed access for analytical and reporting applications.

Storage systems receive processed device monitoring data and organize this information within database architectures that support both real-time access for operational monitoring and historical retention for trend analysis and predictive maintenance applications. The storage infrastructure accommodates various deployment scenarios including local database implementations for individual practice environments and cloud-based systems that support distributed organizations with multiple practice locations. Data organization strategies utilize indexing, partitioning, and archiving techniques that optimize query performance and storage efficiency while maintaining comprehensive historical records that enable long-term analysis of device performance trends and operational patterns. The storage system provides standardized access interfaces that enable various analytical tools, reporting applications, and business intelligence platforms to retrieve device monitoring information through application programming interfaces, direct database connections, or data export mechanisms.

Analytical processing examines stored device monitoring data through various computational methods that identify patterns, trends, and anomalies within device operational records across different time periods and organizational contexts. The analytical system processes device performance metrics, error frequency patterns, and usage characteristics to generate insights regarding device health, maintenance requirements, and operational optimization opportunities that support proactive management of dental imaging equipment. Statistical analysis algorithms examine correlations between device operational variables and performance outcomes to identify predictive indicators that enable early detection of potential issues before complete operational failures occur. Machine learning components analyze historical device data to develop predictive models that forecast maintenance requirements, component replacement needs, and performance degradation patterns based on observed operational characteristics and environmental factors.

Reporting mechanisms generate comprehensive documentation of device operational status, performance trends, and analytical insights through various presentation formats that accommodate different stakeholder information needs and technical capabilities. The reporting system produces real-time dashboards that display current device status across multiple locations, along with historical reports that document performance trends, error patterns, and maintenance activities over extended time periods. Customizable reporting features enable stakeholders to configure analytical parameters, filtering criteria, and presentation formats according to specific operational requirements and organizational preferences. The reporting infrastructure supports multiple access methods including web-based interfaces, mobile applications, and programmatic interfaces that enable integration with external business intelligence tools and practice management systems.

Notification systems monitor processed device data for conditions that warrant stakeholder attention and generate alert messages through various communication channels including email, text messaging, and application-based push notifications. The notification processing evaluates device status information against configurable criteria that define error severity levels, operational impact assessments, and urgency classifications that determine appropriate notification actions and recipient selection. Alert generation procedures format notification content according to communication channel requirements while including comprehensive device identification, error descriptions, and recommended response actions that enable effective stakeholder response to detected issues. The notification system tracks message delivery status and provides confirmation mechanisms that ensure stakeholders receive device status information despite potential communication disruptions or device availability limitations.

Troubleshooting capabilities utilize accumulated device monitoring data to provide diagnostic assistance and resolution guidance when operational issues are detected during device monitoring activities. The troubleshooting system correlates current error conditions with historical patterns and resolution procedures to generate specific recommendations for addressing detected problems based on device characteristics, operational context, and proven solution strategies. Automated diagnostic algorithms analyze error symptoms, device specifications, and environmental factors to identify probable causes and suggest appropriate corrective actions that address both immediate operational issues and underlying conditions that may contribute to recurring problems. The troubleshooting system maintains comprehensive knowledge bases that document resolution procedures for common device issues while incorporating field experience and manufacturer guidance to provide effective problem-solving resources for stakeholders with varying technical expertise levels.

Integration capabilities enable the monitoring system to connect with external analytical platforms, business intelligence tools, and practice management systems through standardized interfaces that support data sharing and collaborative analysis activities. The integration framework provides application programming interfaces that enable external systems to access device monitoring data while maintaining security and access control requirements that protect sensitive operational information. Data synchronization mechanisms support real-time or scheduled data transfer operations that maintain consistency between the monitoring system and external analytical platforms while accommodating different data formats and communication protocols. The integration architecture supports modular expansion that enables connection with emerging analytical tools and specialized applications that may provide enhanced capabilities for specific analytical or operational requirements.

Scalability features enable the monitoring system to accommodate growing device populations and expanding organizational requirements through distributed processing architectures and flexible deployment options that maintain performance and reliability as operational demands increase. The scalable infrastructure supports horizontal expansion through additional processing nodes and storage capacity that can be deployed incrementally to match organizational growth patterns and usage requirements. Load balancing mechanisms distribute monitoring workloads and analytical processing operations across available system resources to maintain consistent response times and operational reliability despite varying usage patterns and peak demand periods. The scalable architecture accommodates multi-tenant deployments that serve multiple organizations simultaneously while maintaining data isolation and security boundaries between different customer environments.

### Example Computing Device

As discussed above, functions relating to interfaces and monitoring the health of dental imaging devices of the subject disclosure can be performed with the use of one or more computing devices. The computing device may include at least a central processing unit (CPU) or other processor device, random access memory (RAM) and/or read only memory (ROM). Depending on the type of computing device, the computing device may include a keyboard (physical or digital via user interface (UI)), a mouse or mouse function via UI, and a communication interface.

As will be appreciated by one skilled in the art, aspects of the disclosed invention may be embodied as a system, method or process, or computer program product. Accordingly, aspects of the disclosed invention may take the form of an entirely hardware embodiment, an entirely software embodiment (including firmware, resident software, micro-code, etc.) or an embodiment combining software and hardware aspects that may all generally be referred to herein as a "circuit," "module," or "system." Furthermore, aspects of the disclosed invention may take the form of a computer program product embodied in one or more computer readable media having computer readable program code embodied thereon.

Any combination of one or more computer readable media may be utilized. In the context of this disclosure, a computer readable storage medium may be any tangible or non-transitory medium that can contain or store a program for use by or in connection with an instruction execution system, apparatus, or device. A computer readable storage medium may be, for example, but not limited to, an electronic, magnetic, optical, electromagnetic, infrared, or semiconductor system, apparatus, or device, or any suitable combination of the foregoing.

Aspects of the disclosed invention are described above with reference to block diagrams of methods, apparatus (systems) and computer program products according to embodiments of the invention. It will be understood that each block of the block diagrams, and combinations of blocks in the flowchart illustrations and/or block diagrams, can be implemented by computer program instructions. These computer program instructions may be provided to the processor of the computing device, which executes via the processor means for implementing the functions/acts specified in the flowchart and/or block diagram block or blocks.

The previous description is provided to enable any person skilled in the art to practice the various aspects described herein. The previous description provides various examples of the subject technology, and the subject technology is not limited to these examples. Various modifications to these aspects will be readily apparent to those skilled in the art, and the generic principles defined herein may be applied to other aspects. Thus, the claims are not intended to be limited to the aspects shown herein, but is to be accorded the full scope consistent with the language claims, wherein reference to an element in the singular is not intended to mean “one and only one” unless specifically so stated, but rather “one or more.” Unless specifically stated otherwise, the term “some” refers to one or more. Pronouns in the masculine (e.g., his) include the feminine and neuter gender (e.g., her and its) and vice versa. Headings and subheadings, if any, are used for convenience only and do not limit the invention.

A phrase such as an “aspect” does not imply that such aspect is essential to the subject technology or that such aspect applies to all configurations of the subject technology. A disclosure relating to an aspect may apply to all configurations, or one or more configurations. An aspect may provide one or more examples. A phrase such as an aspect may refer to one or more aspects and vice versa. A phrase such as an “embodiment” does not imply that such embodiment is essential to the subject technology or that such embodiment applies to all configurations of the subject technology. A disclosure relating to an embodiment may apply to all embodiments, or one or more embodiments. An embodiment may provide one or more examples. A phrase such an embodiment may refer to one or more embodiments and vice versa. A phrase such as a “configuration” does not imply that such configuration is essential to the subject technology or that such configuration applies to all configurations of the subject technology. A disclosure relating to a configuration may apply to all configurations, or one or more configurations. A configuration may provide one or more examples. A phrase such a configuration may refer to one or more configurations and vice versa.

The word “exemplary” is used herein to mean “serving as an example or illustration.” Any aspect or design described herein as “exemplary” is not necessarily to be construed as preferred or advantageous over other aspects or designs.

CLAIMS

1. A system for monitoring and reporting the health of dental imaging devices, comprising:
an interface configured to integrate with one or more application programming interfaces (APIs);
a monitoring module configured to capture and log device status codes, error codes, device location, and operational data during use of the dental imaging devices;
a database configured to store the captured and logged data;
a backend server configured to process status data received from the monitoring module and generate processed data for storage in the database; and
a reporting module configured to provide stakeholders with insights into operational health of the dental imaging devices based on the processed data stored in the database and display the insights on a user interface of an electronic display.

2. The system of claim 1, wherein the monitoring module is further configured to capture contextual information comprising a signed-in user operating one of the dental imaging devices, an identifier of a PC to which the dental imaging device is connected, a serial number of the dental imaging device, and a type and criticality of detected errors found on the dental imaging device.

3. The system of claim 2, wherein the contextual information further comprises a full stack trace of any errors encountered and detailed hints for resolving detected errors.

4. The system of claim 1, wherein the dental imaging devices comprise at least one of intraoral X-ray sensors, intraoral cameras, panoramic X-ray machines, cephalometric X-ray machines, cone beam computed tomography (CBCT) machines, or intraoral scanners.

5. The system of claim 1, further comprising a troubleshooting module configured to utilize the logged data to assist stakeholders in diagnosing and resolving device issues by providing detailed error context and guidance for resolving known errors.

6. The system of claim 5, wherein the troubleshooting module is configured to provide automated suggestions for resolving common device issues based on analysis of the logged data and historical error patterns.

7. The system of claim 1, wherein the database is configured to be hosted locally or in a cloud environment and is capable of storing historical data for long-term analysis of device health and performance trends.

8. A method of monitoring and reporting the health of dental imaging devices, comprising:
integrating a monitoring system with dental imaging devices using one or more application programming interfaces (APIs);
capturing and logging operational data including status codes, error codes, user interactions, device location, and device metadata during operation of the dental imaging devices;
transmitting the captured operational data to a backend server;
processing the operational data at the backend server to generate standardized data;
storing the standardized data in a database;
analyzing the stored data; and
providing insights into device health and providing troubleshooting information on a user interface.

9. The method of claim 8, wherein the dental imaging devices comprise at least one of intraoral X-ray sensors, intraoral cameras, panoramic X-ray machines, cephalometric X-ray machines, cone beam computed tomography (CBCT) machines, or intraoral scanners.

10. The method of claim 8, wherein capturing and logging operational data further comprises capturing contextual information including a signed-in user operating the device, an identifier of a PC to which the device is connected, and a serial number of the device.

11. The method of claim 10, wherein the contextual information further comprises a full stack trace of any errors encountered and detailed hints for resolving detected errors.

12. The method of claim 8, wherein analyzing the stored data comprises correlating current error conditions with historical patterns to generate specific recommendations for addressing detected problems.

13. The method of claim 12, wherein the specific recommendations include automated suggestions for resolving common device issues based on device characteristics and proven solution strategies.

14. The method of claim 8, further comprising a step of generating real-time notifications to stakeholders when critical errors are detected during the capturing and logging of operational data.

15. A non-transitory computer-readable medium storing instructions that, when executed by a processor, cause the processor to perform operations comprising:
integrating a monitoring system with dental imaging devices using one or more application programming interfaces (APIs);
capturing and logging operational data including status codes, error codes, user interactions, device location, and device metadata during operation of the dental imaging devices;
transmitting the captured operational data to a backend server;
processing the operational data at the backend server to generate standardized data;
storing the standardized data in a database;
analyzing the stored data; and
providing insights into device health and providing troubleshooting information on a user interface.

16. The non-transitory computer-readable medium of claim 15, wherein the dental imaging devices comprise at least one of intraoral X-ray sensors, intraoral cameras, panoramic X-ray machines, cephalometric X-ray machines, cone beam computed tomography (CBCT) machines, or intraoral scanners.

17. The non-transitory computer-readable medium of claim 15, wherein the contextual information comprises a signed-in user operating one of the dental imaging devices, an identifier of a PC to which the dental imaging device is connected, a serial number of the dental imaging device, and a location of the dental imaging device.

18. The non-transitory computer-readable medium of claim 17, wherein the contextual information further comprises a full stack trace of any errors encountered and detailed hints for resolving detected errors on the dental imaging device.

19. The non-transitory computer-readable medium of claim 15, wherein the operations further comprise generating real-time notifications to stakeholders when critical errors are detected during the monitoring of operational status.

20. The non-transitory computer-readable medium of claim 18, wherein the operations further comprise correlating current error conditions with historical patterns stored in the database to generate automated suggestions for resolving common device issues.
